(19) United States
(12) Patent Application Publication
(10) Pub. No.: US 2007/0129803 A1
(43) Pub. Date: Jun. 7, 2007

(54) Accommodative Intraocular Lens

(75) Inventors: J. Stuart Cumming, Laguna Beach, CA (US); J. Andy Corley, Laguna Hills, CA (US); Richard L. Lindstrom, Minneapolis, MN (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: C&C Vision International Limited

(21) Appl. No.: 11/549,818

(22) Filed: Oct. 16, 2006

Related U.S. Application Data
(62) Division of application No. 11/295,924, filed on Dec. 6, 2005, now abandoned.

(51) Int. Cl. A61F 2/16

(52) U.S. Cl. 623/6.28; 623/6.37; 623/6.44

(57) ABSTRACT

An accommodating intraocular lens where the optic is moveable relative to the outer ends of the extended portions. The lens comprises an optic made from a flexible material combined with extended portions that is capable of multiple flexions without breaking. The optic has a central area of increased power of less than 1.0 diopter aid near vision. A method is disclosed of implanting the present lens in the non-dominant eye of a patient.

### CROSS REFERENCE TO RELATED APPLICATIONS

This application is a divisional of application Ser. No. 11/295,924 filed on Dec. 6, 2005 which is expressly incorporated herein by reference.

### BACKGROUND

Intraocular lenses have for many years had a design of a single optic with loops attached to the optic to center the lens and fixate it in the empty capsular bag of the human lens. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990s an acrylic material was introduced as the optic of lenses. The acrylic lens comprised a biconvex optic with a straight edge into which were inserted loops to center the lens in the eye and fixate it within the capsular bag.

Recently accommodative or accommodating intraocular lenses have been introduced to the market, which generally are modified plate haptic lenses and, like the silicone plate haptic lenses, have no clear demarcation between the junction of the plate with the optic's posterior surface. A plate haptic lens may be referred to as an intraocular lens having two or more plate haptics joined to the optic.

Flexible acrylic material has gained significant popularity among ophthalmic surgeons. In 2003 more than 50% of the intraocular lenses implanted had acrylic optics. Hydrogel lenses have also been introduced. Both the acrylic and hydrogel materials are incapable of multiple flexions without fracturing.

The advent of an accommodating lens which functions by moving along the axis of the eye by repeated flexions somewhat limited the materials from which the lens could be made. Silicone is the ideal material, since it is flexible and can be bent probably several million times without showing any damage. Additionally a groove or hinge can be placed across the plate adjacent to the optic as part of the lens design to facilitate movement of the optic relative to the outer ends of the haptics. On the other hand, acrylic material fractures if it is repeatedly flexed.

### SUMMARY OF THE INVENTION

According to a preferred embodiment of this invention, an accommodating lens comprises a lens with a flexible solid optic attached to which are two or more extended portions which may be plate haptics capable of multiple flexions without breaking, preferably along with fixation and centration features at their distal ends. There may be a hinge or groove across the extended portions adjacent to the optic to facilitate the anterior and posterior movement of the optic relative to the outer ends of the extended portions.

Importantly, the center of the optic of the lens of the present invention has a central area of less than 1.0 diopter to aid in near vision. Preferably, the accommodating lens is to be implanted in the patient's non-dominant eye to provide improved instant near vision.

Thus, the present invention is directed to an accommodating lens with the increased power central area, and a method wherein a conventional accommodating lens, such as the type disclosed in U.S. Pat. No. 6,387,126 and others in the name of J. Stuart Cumming, is implanted in the dominant eye of the patient, and the lens of the present invention having the increased power central area is implanted in the non-dominant eye.

Accordingly, features of the present invention are to provide an improved form of accommodating lens including a central area of increased power, and a method of implanting that type of lens in a patient's non-dominant eye and implanting a conventional accommodating lens in the dominant eye.

### BRIEF DESCRIPTION OF THE DRAWINGS

FIG. 1 is a perspective view of a preferred embodiment of the present invention.

FIG. 2 is a front elevational view.

FIG. 3 is a side elevational view.

FIG. 4 is an end view.

FIG. 5 illustrates the lens, showing T-shaped haptics engaged in the capsular bag having been depressed by the bag wall toward the optic.

FIGS. 6a and 6b provide details of the blended design transition of the anterior optic surface from the outside to the center of the lens.

According to the present invention the optic is of a foldable, flexible silicone, acrylic or hydrogel material and the haptic plates are of a foldable material that will withstand multiple foldings without damage, e.g., silicone. Preferably, the end of the plate haptics have T-shaped fixation devices and are hinged to the optic.

### DESCRIPTION OF THE PREFERRED EMBODIMENT

Turning now to the Figures, a preferred embodiment is illustrated in detail comprising an intraocular lens 1 formed as a flexible solid optic 2 preferably made of silicon, and flexible extending portions 4 of any suitable form which may be plate haptics or fingers which are capable of multiple flexations without damage and formed, for example, of silicone. The optic 2 and haptics 4 preferably are uniplanar, and one or more haptics 4 extend distally from opposite sides of the optic 2.

According to the present invention, the optic 2 has a central blended area 3. The lens 1 preferably comprises an accommodating intraocular lens currently available from eyeonics, inc., Aliso Viejo, Calif., such as shown in U.S. Pat. No. 6,387,126, typically with a 4.5 mm diameter optic, but with a 1.0 to 2.5 mm diameter central area 3 and which has an added of less than 1 dioptor of power in the center of the lens 1. The area 3 is on the anterior side of the lens, and the posterior side can be any conventional form or can be toric if desired, or just the posterior surface behind the bulls eye could be toric. The added power area 3 is to aid in near vision. The optic diameter can range from approximately 3.5 to 8.0 mm but a typical one is 4.5 mm.

Non-accommodating intraocular lenses have been disclosed with a central area with a power of 2.0 diopters or more. Examples are in Nielson, U.S. Pat. No. 4,636,211, and Keats, U.S. Pat. No. 5,366,500. Such lenses result in the patient having two separate images, although the brain tends to ignore an unwanted image.

Importantly, with the present accommodating lens having a central area of less than 1.0 diopter the distant vision of the patient will slightly blur with no separate images, but also improve the near vision principally through an increased depth of field. Thus, there will not be two separate images, but a blurred primary image which when seen in one eye only, preferably with the other eye having a standard intraocular lens, is believed to essentially be not noticeable by the patient.

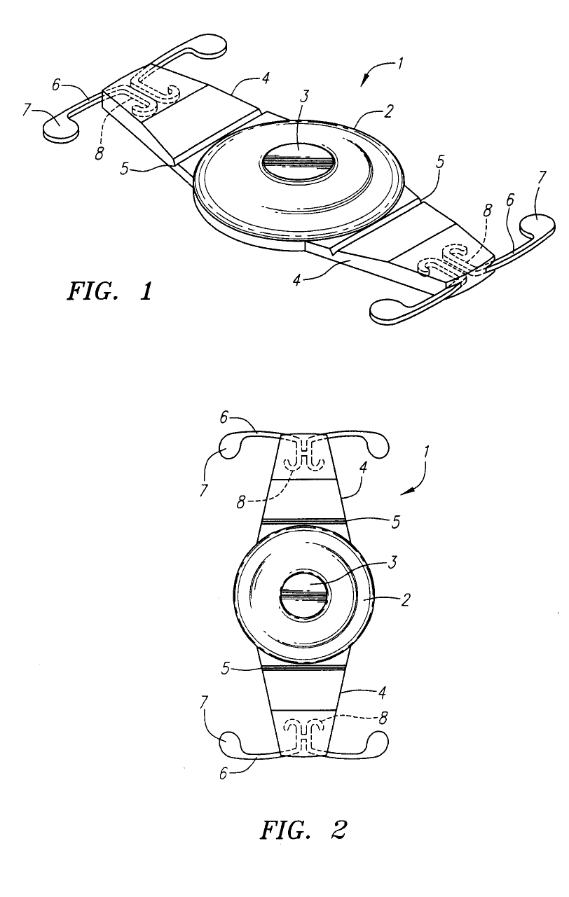

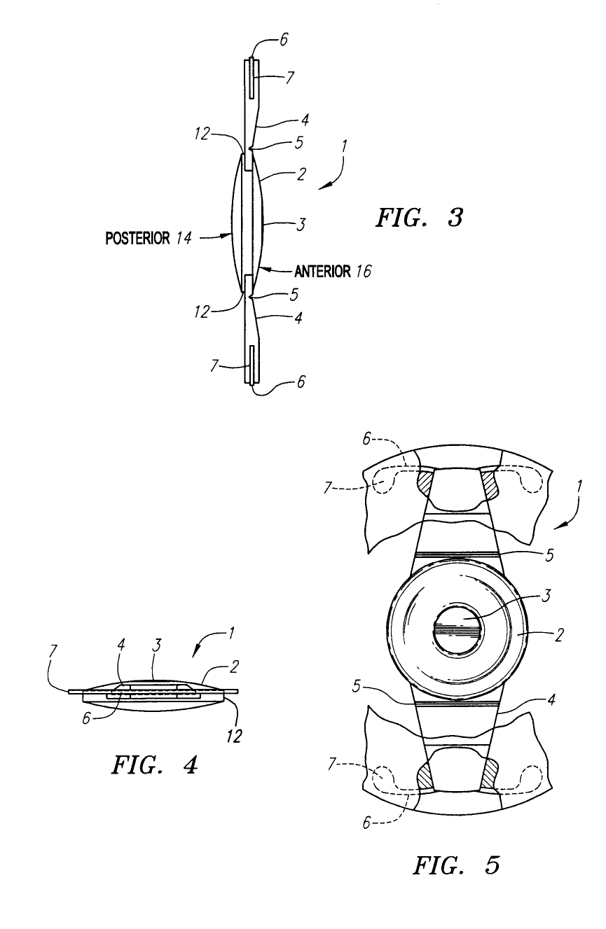

The haptics preferably are plate haptics having arcuate outer edges including loops 6. The loops 6 when unrestrained are somewhat less curved in configuration as shown in FIGS. 1-2, but compare an example of an inserted lens 1 as seen in FIG. 5. The lens 1, including the optic 2, haptics 4, and loops 6 is preferably formed of a semi-rigid material such as silicone, acrylic, or hydrogel, and particularly a material that does not fracture with time. The loops 6 can be of a material different from the haptics 4 and retained in the haptics by loops 8 molded into the ends of the haptics. Grooves or thin areas 5 forming hinges preferably extend across the haptics 4 adjacent to the optic 2.

The flexible haptics 4 and loops 6 can be connected to an acrylic optic 2 by means of an encircling elastic band (not shown) which fits into a groove in the acrylic optic 2 as shown and described in co-pending application Ser. No. 10/888,536 filed Jul. 8, 2004 and assigned to the assignee of the present application.

There can be a sharp edge 12 around the posterior surface 14 of the optic 2. The junction of the posterior surface 14 of the optic 2 to the edge of the lens 1 is a sharp edge or junction 12 designed to reduce the migration of cells across the posterior capsule of the lens post-operatively and thereby reduce the incidence of posterior capsular opacification and the necessity of YAG posterior capsulotomy. The anterior surface 16 of the optic 2 is closer to the groove 2 than is the posterior surface 14.

FIG. 1 illustrates the haptics 4, loops 6, hinge 5 across the haptics adjacent to the optic 2. Hard knobs 7 can be provided on the ends of the loops 6 and are designed to fixate the loops 6 in the capsular bag of the eye and at the same time allow the loops 6 to stretch along their length as the optic 2 of the lens 1 moves backward and forward and the haptics 4 move or slide within pockets formed between the fusion of the anterior and posterior capsules of the capsular bag.

The present bulls eye concepts are applicable to several forms of lenses, such as lenses shown in Cumming U.S. Pat. Nos. 5,476,514, 6,051,024, 6,193,750, and 6,387,126.

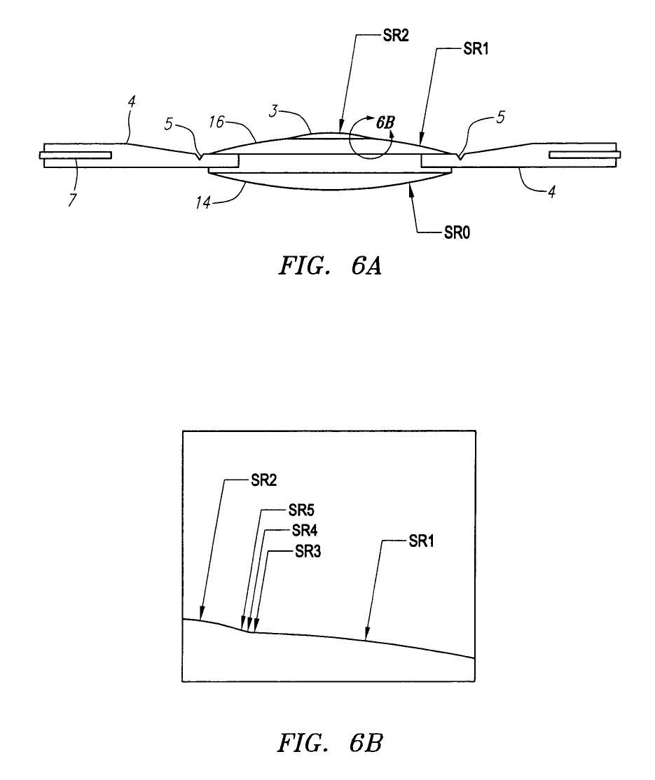

FIGS. 6a and 6b illustrate more detail of the blended design of the anterior optic surface 16 and thus show the transition of the anterior optic surface from the outside surface of spherical radius SR1 to the center surface of the spherical radius of SR2 which comprises the central area 3 illustrated in the other Figures. FIGS. 6a and 6b demonstrate the transition area as a varying radius that ranges from SR1 to SR2, and it should be noted that the difference between SR1 and SR2 has been enhanced to better show the transition. In particular, SR1 is >SR3>SR4>SR5>SR2.

As is well known in the art, the intraocular lens 1 such as that in the drawings is implanted in the capsular bag of the eye after removal of the natural lens. The lens is inserted into the capsular bag by a generally circular opening cut in the anterior capsular bag of the human lens and through a small opening in the cornea or sclera. The outer ends of the haptics 4, or loops 6, are positioned in the cul-de-sac of the capsular bag. The outer ends of the haptics, or the loops, are in close proximity with the bag cul-de-sac, and in the case of any form of loops, such as 6, the loops are deflected from the configuration as shown for example in FIG. 2 to the position shown in FIG. 5. The knobs 7 can be provided on the outer end portions of the loops 6 for improved securement in the capsular bag or cul-de-sac by engagement with fibrosis, which develops in the capsular bag following the surgical removal of the central portion of the anterior capsular bag. Additionally, according to the present invention, the lens with the central area 3 is intended to be implanted in the non-dominant eye of the patient, and a conventional interocular lens like that seen in the drawings but without the central area 3 is intended to be implanted in the dominant eye of the patient. The present lens implanted in the non-dominant eye is intended to give superior instant near vision than if the non-dominant eye has implanted therein a lens without the central area 3. The lenses are implanted in the same manner as described above and as known in the art.

There are two descriptions of central diopter and range that should be considered.
The first looks at the distribution of the lens over the dioptric power range of 4.0 to 33.0, the mode—or the most commonly used dioptric power of the lens is 22.0 diopter.
A histogram of the lens is basically a bell curve with a peak at 22.0 diopter. Often analysis is done with a 22 diopter lens for this very reason.
The second can be relative to the lens design with the central diopter being the dioptric power of the center portion 3 of the lens of typically 1.5 mm diameter. The dioptric power of this area will be <1.0 larger than that of the surrounding area—thus the <1.0 diopter add region.

The lens design is sewed on the existing eyeonics Crystalens to the extent of the following:
Lens and plate haptics are manufactured from the same mold; however, one of the pins for molding the anterior optical surface of the present lens is different.
Lens and plate material is Biosil (Silicone).
Haptic is the same design.
Haptic material is the same Kapton HN (polyimide).
The posterior surface SR0 may be the same as or different than SR1 (e.g. a 23 diopter pin on the anterior side and a 21 diopter pin on the posterior side will give a 22 diopter lens).

Below are calculated dimensions of the optical section of the IOL for the minimum, average and maximum diopter lens. Diopter 1 is the dioptric power through the outer perimeter of the lens, and Diopter 2 is through the center section. Note that the radii are approximate as SR0 (posterior surface spherical radius) and SR1 (anterior surface spherical radius—outer area) aren't necessarily the same. The center thickness on the center area 3 is approximately 3 microns (0.003 mm) thicker over the 4 to 33 diopter range.

| | | SR0 & | | Center |
|---|---|---|---|---|
| | | SR1 | SR2 | Thickness |
| Diopter 1 | Diopter 2 | (mm) | (mm) | (mm) |
| 4 | 5 | 45.47 | 30.30 | 0.46 |
| 22 | 23 | 8.24 | 7.55 | 0.97 |
| 33 | 34 | 5.47 | 5.16 | 1.32 |

After the lens is manufactured, it is tumbled with a slurry of glass beads to remove any flashing, smooth the edges and integrate the radii, and it shrinks, resulting in an absence of discrete radii SR1-SR5, and thus ends up not a multiple power lens. The resulting blended design after completion does not cause separate images as does a multifocal lens, but actually provides a central curve which provides additional focusing power and actually results in an extended region of depth of field about the far point of the patient's vision. Thus, a desired depth of field increase about the focal point occurs, and the retinal image has been determined to be superior over a wider range than a standard accommodating intraocular lens. The through focus wavefront aberrations peak to valley and RMS graphs and Waveforms 1 and 2 below show quantitatively how the present ED-AIOL provides superior overall optical performance in the range of object vergence from infinity to 2 D. Thus, the lens functions simply by extending the range of accommodation about the far point by increasing the static depth of field. A patient's vision is improved by virtue of an increased depth of field, and this depth of field also will be present if the patient wears spectacles for near vision.

[image]

The Waveforms 1 are peak to valley wavefront aberrations for AIOL and ED-AIOL forobject vergence distance from 0 D (object at infinity) to 2 D (500 mm).

[image]

The Waveforms 2 are RMS wavefront aberrations for AIOL and ED-AIOL for object vergence distance from 0 D (object at infinity) to 2 D (500 mm).

In the Waveforms 1 and 2 it can be seen that the AIOL provides lower wavefront aberration errors in terms of peak to valley and RMS values over the rage of object distance from infinity to about 4 M (0.25 D). For closer object distances (4 M to 500 mm), the ED-AIOL provides better optical performance. In the majority of the object vergence range, the ED-AIOL provides about 33% better P-V performance and about 50% better RMS performance compared to the AIOL. As can be seen from the lateral shift in the graphs, this corresponds to about a 0.3 D improvement for the ED-AIOL. This again demonstrates the fact that the ED-AIOL should provide better overall performance over the depth of field range about the AIOL's focal point.

The end of the loops 6 containing the knobs 7 may be either integrally formed from the same material as the haptics 4 or the loops may be of a separate material such as polyimide, prolene, or PMMA as discussed below. The loops if formed of a separate material are molded into the terminal portions of the haptics 4 such that the flexible material of the loop 6 can extend by elasticity along the internal fixation member of the loop.

As noted above, the haptics 4 may have a groove or thin area 5 forming a hinge across their surface adjacent to the optic. This facilitates movement of the optic anteriorly and posteriorly relative to the outer ends of the haptics.

Accordingly, there has been shown and described a lens that ideally comprises a silicon optic and silicone haptic plates, loops that can be of a different material than the plate, and a fixation device at the end of each loop allowing for movement of the loops along the tunnel formed in the fusion of the anterior and posterior capsules of the human capsular bag, and wherein the anterior surface of the optic has a central area of increased power of less than 1 diopter as well as a method of implanting the lens in the non-dominant eye.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

What is claimed is:

1. A method for improving near vision of a non-dominant eye of a patient comprising the steps of
implanting in the non-dominant eye of the patient an accommodating intraocular lens which has a flexible lens body having normally anterior and posterior sides and including a flexible solid optic, the optic having a central area of increased power of less than 1.0 diopter to enable an extended region of depth of field about the far point of a patient's vision, the lens body having two or more extending portions from the optic such that the lens can move anteriorly with contraction of the cilary muscle of the eye, and the lens being sized to be implanted into the capsular bag of the eye such that contraction of the ciliary muscle causes the optic of the lens within the capsular bag behind the iris to move forward to toward the iris with its contraction.

2. A method as in claim 1 comprising the further steps of
implanting in the dominant eye of the patient an accommodating intraocular lens which has a flexible lens body having normally anterior and posterior sides and including a flexible solid optic, the lens body having two or more radially extending portions from the optic such that the optic of the lens can move anteriorly with contraction of the cilary muscle of the eye.

3. Accommodating intraocular lenses for implantation in the eyes of a patient comprising two flexible lens bodies having normally anterior and posterior sides, each including a flexible solid optic,
the lens bodies each having two or more radially extending portions from each optic such that the optic of the lenses can move anteriorly with contraction of the ciliary muscles of the eye,
one optic having a central area of increased power of less than 1.0 diopter on the anterior side of the optic, and
each lens being sized to be implanted into a respective capsular bag of the eye such that contraction of the ciliary muscles causes the optics of the lenses within the capsular bags behind the iris to move forward toward the iris with muscle contraction.

4. Accommodating lenses according to claim 3, wherein the extending portions are plate haptics.

5. Accommodating lenses according to claim 3, wherein the extending portions are plate haptics with a narrowing of the plate junctions adjacent to the optic.

6. Accommodating lenses according to claim 3, wherein the lens having the optic with a central area of increased power is to be implanted in a non-dominate eye of a patient.
